(19) United States
(12) Patent Application Publication
(10) Pub. No.: US 2023/0211461 A1
(43) Pub. Date: Jul. 6, 2023

(54) SANDBLASTING SURFACE TREATMENT METHOD FOR CHANGE IN SURFACE MORPHOLOGY AND IMPROVEMENT IN RESIDUAL STRESS OF THREE TYPES OF DENTAL ZIRCONIA

(71) Applicant: AJOU UNIVERSITY INDUSTRY-ACADEMIC COOPERATION FOUNDATION, Gyeonggi-do (KR)

(72) Inventor: Hee-Kyung KIM, Seoul (KR)

(73) Assignee: AJOU UNIVERSITY INDUSTRY-ACADEMIC COOPERATION FOUNDATION, Gyeonggi-do (KR)

(21) Appl. No.: 18/071,407

(22) Filed: Nov. 29, 2022

(30) Foreign Application Priority Data
Nov. 30, 2021 (KR) 10-2021-0169406

(51) Int. Cl. B24C 1/08; C04B 35/48

(52) U.S. Cl. CPC B24C 1/08; C04B 35/48; C04B 2235/3246

(57) ABSTRACT

The present invention relates to a surface treatment method for dental zirconia, including sandblasting the surface of three types of dental zirconia (3Y-TZP, 4Y-PSZ and 5Y-PSZ) with alumina particles, and this method optimizes sandblasting conditions for each type of zirconia and enhances mechanical properties by strengthening residual stress. In addition, a dental article including dental zirconia made by the surface treatment method for zirconia, and suitable protocols for the durable bond between resin cements and high-translucent zirconia are suggested.

### CROSS-REFERENCE TO RELATED APPLICATION

This application claims priority to and the benefit of Korean Patent Application No. 2021-0169406, filed on Nov. 30, 2021, the disclosure of which is incorporated herein by reference in its entirety.

### BACKGROUND

### 1. Field of the Invention

The present invention relates to a surface treatment method for zirconia of dental ceramics, suggesting optimized protocols for sandblasting three types of dental zirconia with alumina particles. Accordingly, this method changes surface morphology and improves residual stress, allowing various applications of dental zirconia due to improved mechanical properties.

### 2. Discussion of Related Art

Structural ceramics have been applied in dentistry to replace metallic dental prostheses due to a growing interest in esthetics and growing concern over metal allergies. Among dental ceramics, zirconia ceramics are rapidly attracting attention due to their excellent mechanical properties, high biocompatibility and relative esthetic potential. Zirconia exhibits three phases according to a temperature: e.g., the monoclinic phase at 1170° C. or less, the tetragonal phase at 1170 to 2370° C., and the cubic phase at 2370° C. or more. Since a volume change of approximately 3 to 5% accompanies the phase transformation process from the tetragonal phase to the monoclinic phase, a dopant oxide such as yttria (Y₂O₃) is used to stabilize a tetragonal structure at room temperature. Three mol% yttria-stabilized tetragonal phase zirconia polycrystal (3Y-TZP) with a high mechanical strength has been widely used in dentistry.

Although zirconia has been regarded as a promising biomaterial in dentistry, this material has several drawbacks in clinical application. For example, 3Y-TZP has specific optical disadvantages due to a relatively high refractive index, resulting in a high level of light reflection. High reflectance further worsens translucency and esthetics. Therefore, recent developments have focused on improvement in the translucency of 3Y-TZP particularly as a monolithic (fully anatomical) system for dental restorations. More recently, novel high-transparent zirconia was introduced by increasing the amount of a stabilization oxide (Y₂O₃) to stabilize a cubic structure at room temperature. The degraded mechanical properties of the novel high-transparent zirconia are generally considered as a main weakness, but the improved transparency of a cubic phase containing partially stabilized zirconia ceramics (4 mol% partially stabilized zirconia (4Y PSZ) and 5 mol% partially stabilized zirconia (5Y-PSZ)) make it possible to fabricate esthetic restorations without an additional veneering technique.

Another potential concern with zirconia restorations is the bonding problem between resin cement and zirconia, which is associated with clinical reliability. Since zirconia is a non-silica-based ceramic, the zirconia surface cannot be etched with hydrofluoric acid. The retention of zirconia restorations depends on the mechanical roughness of the surface and chemical bonding with acidic adhesive monomers such as 10-methacryloxydecyl dihydrogen phosphate (MDP). The most commonly used mechanical pretreatment to promote bonding between resin cement and zirconia is sandblasting of the inner surface of a zirconia restoration with aluminum oxide (Al₂O₃) particles. The sandblasting technique provides a clean and rough surface for micromechanical interlocking. In addition, sandblasting is effective in increasing a surface area, surface wettability, and surface energy. However, bonding performance is highly dependent on sandblasting conditions such as the size and shape of abrasive particles, air pressure, working time, a distance and an impact angle. Sandblasting tends to cause tetragonal-to-monoclinic phase transformation of conventional high-strength zirconia (3Y-TZP), and thereby the transformation strengthening mechanism may increase mechanical strength. However, excessive sandblasting may damage the zirconia surface and lower the flexural strength of 3Y-TZP.

Surface topography may affect the geometrical, physical and chemical properties of a material. For the measurement of surface topography, quality control and the prediction of the functional properties of the surface are critical factors. In surface texture analysis, area-based 3D analysis defined by ISO 25178 may provide more precise information than 2D profile measurement. In many studies, characterization methods for surface topography were investigated. A recently introduced method, feature-based characterization, offers spatial information related to the orientation of topographical properties of the surface. Another systematic approach is a multi-scale analysis that includes the characterization of surface topography at multiple observation scales in order to control various interactions between the scales. A novel 3D motif method may be applied to determine geometrical characteristics such as holes and valleys created by sandblasting. Reflectance confocal microscopy based on a focus detection method is one optical metrological technique. A non-contact optical measurement method has been widely used due to its high reliability and flexibility, but the noise from a light source may affect measurement quality. A novel spiral-scanning laser differential confocal measurement method helps reduce existing disturbance.

Since the accurate topographical measurement of abrasive particles affects the efficiency of a machining process, many studies have suggested sets of parameters for the quantitative evaluation of surface geometry. The volumetric difference during t→m phase transformation may generate compressive residual stress on the zirconia surface, and such residual stress is advantageous to increasing the flexural strength of 3Y-TZP. Confocal Raman spectroscopy using a laser microprobe has been widely used to non-destructively quantify the residual stress on the zirconia surface. The induced residual stress caused phonon deformation of Raman bands, and thus the wavenumber of a zirconia crystal was changed. The amount of band shift may be used to measure residual stress related to the t→m phase transformation. Several studies have revealed that the tetragonal peak at approximately 147 cm⁻¹ shifts to a higher wavenumber, indicating the presence of residual compressive stress. In a recent study, stress-induced Raman peak shifts of the metastable tetragonal phase of stabilized yttria zirconia at 147, 465, 610 and 640 cm⁻¹ in high-temperature annealing were measured, and the Raman peak shift was considered as the structural change in zirconia crystals, and it was reported that internal strain mismatch causes compressive stress, leading to enhanced toughness of a material. High-transparent dental zirconia and conventional zirconia have different phase compositions and microstructures, so they can behave differently from sandblasting. In several studies, the sandblasting effects between different zirconia grades were compared, but these studies were conducted with different sandblasting parameters.

Appropriate sandblasting protocols for a durable bond between resin cement and high-translucent zirconia have not been studied yet. Further, most existing studies on the various effects of alumina sandblasting used only some particle sizes, so the present invention focused on the effect of sandblasting particles with five different sizes on sandblasting performance of dental incorporated zirconia with three different levels of translucency while maintaining other sandblasting parameters constant, and determined the change in sandblasting effect by a function of particle size.

### SUMMARY OF THE INVENTION

As a result of trying to study the optimal protocols by considering a durable bond between resin cement and high-translucent zirconia and a subsurface change after sandblasting, the inventors suggested optimal alumina particle sizes and conditions for sandblasting of three types of dental zirconia, focusing on the effects of five different sizes of sandblasting particles on sandblasting performance for dental zirconia with three different levels of translucency while maintaining other sandblasting parameters constant.

Accordingly, the present invention is directed to providing a surface treatment method for dental zirconia, which includes: (a) polishing the surface of zirconia which mostly includes tetragonal and cubic zirconia with less than 15% monoclinic phase, 95 vol% or more of all particles of which have an average diameter of 100 to 1200 nm, and which has a density of 99.5 % or more of the theoretical density and is opalescent; and (b) sandblasting the polished surface in (a) with alumina particles using a nozzle-equipped sandblasting apparatus.

The present invention is also directed to providing a dental article which includes dental zirconia made by the surface treatment method for dental zirconia.

The terms used herein are used only to explain describe specific examples, not to limit the present invention. Singular expressions include plural referents unless the context clearly indicates otherwise. The terms “include” and “have” used herein designate the presence of characteristics, numbers, steps, operations, components or members described in the specification or a combination thereof, and it should be understood that the possibility of the presence or addition of one or more other characteristics, numbers, steps, operations, components, members or a combination thereof is not excluded in advance. All terms including technical and scientific terms have the same meaning that is generally understood by those skilled in the art unless defined otherwise. General terms, such as terms defined in dictionaries, should be interpreted with meanings according to the context of related technology, and should not be interpreted with ideal or excessively formal meanings unless clearly defined herein.

The present invention is directed to providing a surface treatment method for dental zirconia, which includes: (a) polishing the surface of zirconia which mostly includes tetragonal and cubic zirconia with less than 15% monoclinic phase, 95 vol% or more of all particles of which have an average diameter of 100 to 1200 nm, and which has a density of 99.5% or more of the theoretical density and is opalescent; and (b) sandblasting the polished surface in (a) with alumina particles using a nozzle-equipped sandblasting apparatus.

In one embodiment of the present invention, the zirconia in (a) may be any one selected from the group consisting of 3Y-TZP, 4Y-PSZ and 5Y-PSZ.

In one embodiment of the present invention, in (b), the vertical distance between the nozzle-equipped sandblasting apparatus and the polished surface may be 1 to 100 mm.

In one embodiment of the present invention, in (b), the pressure for sandblasting with alumina particles may be 0.1 to 0.5 Mpa.

In one embodiment of the present invention, when the zirconia is 3Y-TZP, the average particle size of the alumina particles may be 100 to 120 µm.

In one embodiment of the present invention, when the zirconia is 4Y-PSZ, the average particle size of the alumina particles may be 80 to 100 µm.

In one embodiment of the present invention, when the zirconia is 5Y-PSZ, the average particle size of the alumina particles may be 10 to 50 µm.

In one embodiment of the present invention, in the case of the surface treatment method, a target peak at 147 cm⁻¹ shifts to a higher wavenumber than that when sandblasting is not performed.

In one embodiment of the present invention, trace elements of the alumina particles may include Si, Fe and Zr.

In one embodiment of the present invention, the surface treatment method may increase residual stress.

In one embodiment of the present invention, the surface treatment method may cause a greater change in surface topography than that when surface treatment is not performed.

The present invention provides a dental article which includes dental zirconia made by the surface treatment method for dental zirconia.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows the flow chart of an experimental procedure, which illustrates analysis methods related to surface topography, residual stress, microstructure and Al₂O₃ particle analysis, as well as conditions for specimen preparation and Al₂O₃ sandblasting;

FIG. 2 shows the characteristics of alumina particle size distribution, indicating that there was a significant difference between all particle size specifications, and the larger the particle size specification, the larger the measured particle size;

FIG. 3 shows the particle size distributions and scanning electron microscopic images of Al₂O₃ particles;

FIG. 4 shows the 3D morphologies obtained from confocal laser scanning microscope and scanning electron microscope (SEM) images (magnification 10,000×) of each subgroup. In the SEM images, the white arrows indicate microcracks, the red arrows indicate Al₂O₃ particle debris deposited on the polished zirconia surface, the white circles indicate plastic deformation, and the red circles indicate surface melting: (A) 3Y-con.; (B) 3Y-25.; (C) 3Y-50.; (D) 3Y-90.; (E) 3Y-110.; (F) 3Y-125.; (G) 4Y-con.; (H) 4Y-25.; (I) 4Y-50.; (J) 4Y-90.; (K) 4Y-110.; (L) 4Y-125.; (M) 5Y-con.; (N) 5Y-con.; (N) 5Y-25.; (O) 5Y-50.; (P) 5Y-90.; (Q) 5Y-110.; (R) 5Y-125;

FIG. 5 shows surface texture parameters of each subgroup for three different zirconia grades;

FIG. 6 shows the representative µRaman spectra for each subgroup of three different zirconia grades; and

FIG. 7 shows the t-ZrO₂ Raman peak shift at 147 cm⁻¹ for each subgroup of three zirconia grades as a function of Al₂O₃ particle size.

### DETAILED DESCRIPTION OF EXEMPLARY EMBODIMENTS

As described above, the necessity for optimizing suitable sandblasting protocols for a durable bond between resin cements and three types of dental zirconia has emerged. The inventors investigated 3D surface topography using confocal laser scanning microscopy, which is a non-contact optical imaging technique, based on capturing multiple 2D images at different depths that allows reconstruction of a 3D structure through an optical cross-sectional process. In addition, the Raman peak shift at 147 cm⁻¹ was used to characterize residual compressive stresses during sandblasting with various alumina particle sizes, and became a crucial parameter related to the mechanical properties of zirconia. Therefore, the present invention elucidated the effect of the Al₂O₃ sandblasting particle size on the surface morphology and residual stresses of the conventional tetragonal zirconia (3Y-TZP) and two different grades of high-transparent zirconia (4Y-PSZ and 5Y- PSZ). In addition, the present invention provides clinical guidelines for selecting optimal sandblasting particles to achieve the required surface topography with minimal side effects in novel dental zirconia materials.

Hereinafter, the present invention will be described in further detail.

The present invention provides a surface treatment method for dental zirconia, which includes: (a) polishing the surface of zirconia which mostly includes tetragonal and cubic zirconia with less than 15% monoclinic phase, 95 vol% or more of all particles of which have an average diameter of 100 to 1200 nm, and which has a density of 99.5% or more of the theoretical density and is opalescent; and (b) sandblasting the polished surface in (a) with alumina particles using a nozzle-equipped sandblasting apparatus.

The dental zirconia used in the present invention can be used as an esthetic restoration material and exhibits physical properties within the above ranges.

In one embodiment of the present invention, the zirconia may be any one selected from the group consisting of 3Y-TZP, 4Y-PSZ and 5Y-PSZ.

Preferably, when the zirconia in (a) is 3Y-TZP, it may have < 15% cubic phases; when the zirconia in (a) is 4Y-PSZ, it may have > 25% cubic phases; and when the zirconia in (a) is 5Y-PS, it may have > 50% cubic phases.

3 mol% yttria-stabilized tetragonal zirconia polycrystal (3Y-TZP) is 3 mol% yttria-stabilized tetragonal phase zirconia polycrystal with high mechanical strength. 4 or 5 mol% partially stabilized zirconia (4Y-PSZ or 5Y-PSZ) is novel high-transparent zirconia, and compared with traditional zirconia such as 3Y-TZP, its decreased mechanical properties are generally considered as a critical weakness, but its improved transparency allows the fabrication of esthetic restorations without an additional veneering technique.

In one embodiment of the present invention, in (b), the vertical distance between the nozzle-equipped sandblasting apparatus and the polished surface may be 1 to 100 mm, and preferably 5 to 20 mm. Outside the range of the vertical distance, surface morphology may not be changed sufficiently, or residual stress may not be sufficiently induced.

In one embodiment of the present invention, in (b), the pressure for sandblasting with alumina particles may be 0.1 to 0.5 Mpa. To obtain a polished surface, a higher blasting pressure must be used, but outside the above pressure range, surface damage as well as an adverse effect on the adhesion of cements may be caused.

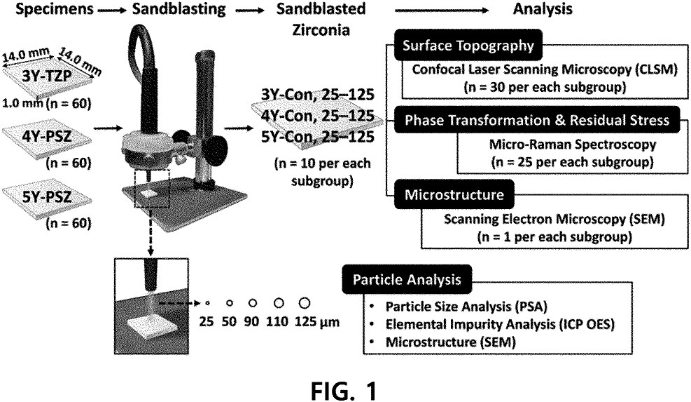

Specifically, in one embodiment of the present invention, as shown in FIG. 1, using the sandblasting apparatus, a zirconia plate was particle-polished 10 mm apart from the tip of the sandblasting apparatus at an impact angle of 90° under pressure of 0.2 MPa for 10 s/cm².

As a result, within the range of sandblasting parameters investigated in the present invention, Al₂O₃ sandblasting changed the surface topography of three different zirconia grades, but each grade showed different responses to changes in particle size that can contribute to the changes in intrinsic physical, chemical and mechanical properties.

In the present invention, phase transformation and related residual stress after sandblasting were evaluated using µRaman spectroscopy. Particularly, by tracking the peak shift of the tetragonal band near 147 cm⁻¹, residual stress in which the tetragonal band and the monoclinic band do not overlap was obtained.

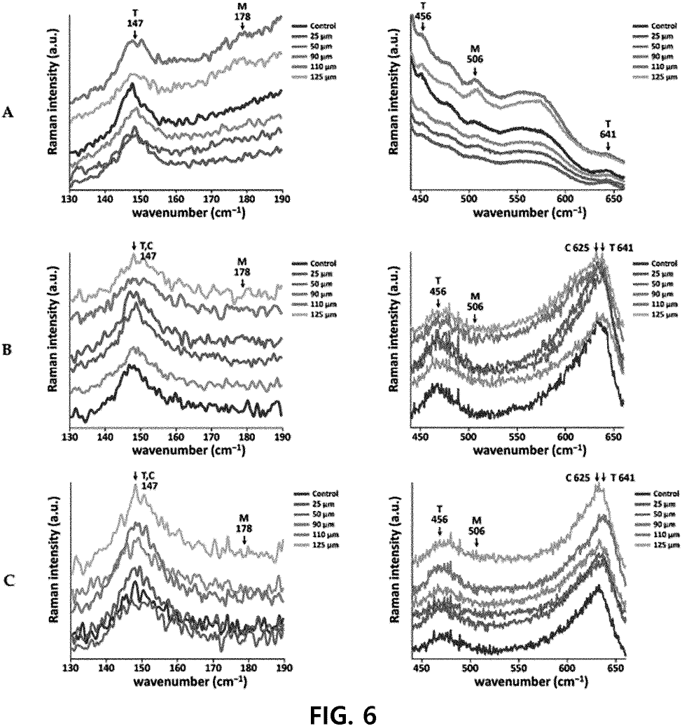

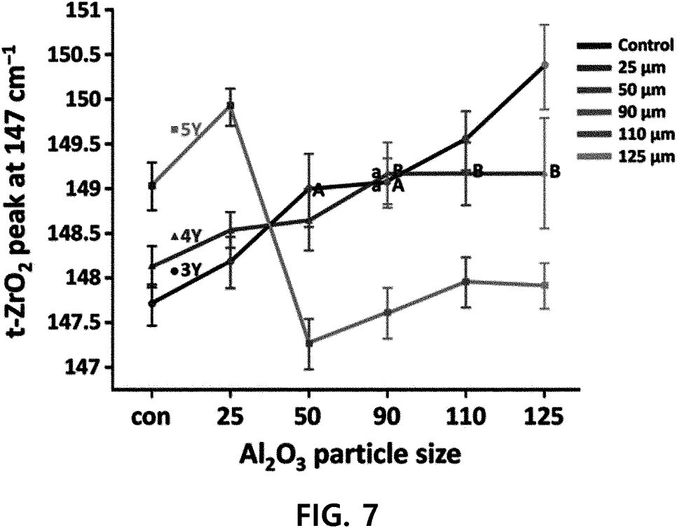

Sandblasting induced tetragonal-to-monoclinic phase transformation on the surface, and thus the related stress shifted up the tetragonal phase band (p < 0.05), which is referred to as a blue shift. As in the previous literature where the Raman shift increases when the material lattice is compressed to gain energy, as shown in FIGS. 6 and 7, in the case of 3Y-TZP, the larger the alumina particles of the present invention, the larger the blue shift (p < 0.05), and as the particle size increased, the tetragonal peak was reduced and the monoclinic phase was increased. A larger blue shift may indicate the presence of greater compressive stress, resulting in improved mechanical properties.

Therefore, according to an exemplary embodiment of the present invention, when the zirconia is 3Y-TZP, the average particle size of alumina particles may be 100 to 120 µm, and preferably, in terms of the given pressure, time, distance and impact angle (0.2 MPa, 10 s/cm², 10 mm and 90°) considering the potential advantages of residual stress related to phase transformation and the risk of mechanical failure, the average particle size of alumina particles may be 110 µm.

In the case of 4Y-PSZ, a broad structure peak was detected at 625 cm⁻¹. There was no relative change in the intensity of the cubic structure peak, but as the particle size increased, the tetragonal peak at 456 cm⁻¹ was reduced. This is related to the low transformability of the cubic phase. Broadened tetragonal peaks having larger particles were also detected at 147 and 456 cm⁻¹, assuming that the broadening of the peaks resulted from the presence of a tetragonal phase or the lattice distortion of tetragonal zirconia as a result of sandblasting. As shown in FIG. 7, the tetragonal peak at 147 cm⁻¹ showed blue shift, and a shift amount increased up to 90 µm (p < 0.05), but no further peak shift was observed (p > 0.05). This is explained by the increased critical stress concentration associated with a crack growth behavior in the residual stress field. The creation of compressive stress enhanced the strength of sandblasted 4Y-PSZ using maximum 90 µm sand, thereby disturbing the propagation of defects. However, the use of larger alumina particles may have an adverse effect on the strength.

Accordingly, according to an exemplary embodiment of the present invention, when the zirconia is 4Y-PSZ, the average particle size of alumina particles may be 80 to 100 µm, and more preferably, to minimize surface defects and obtain a bigger change in surface topography for better bonding, the average particle size of alumina particles may be 90 µm.

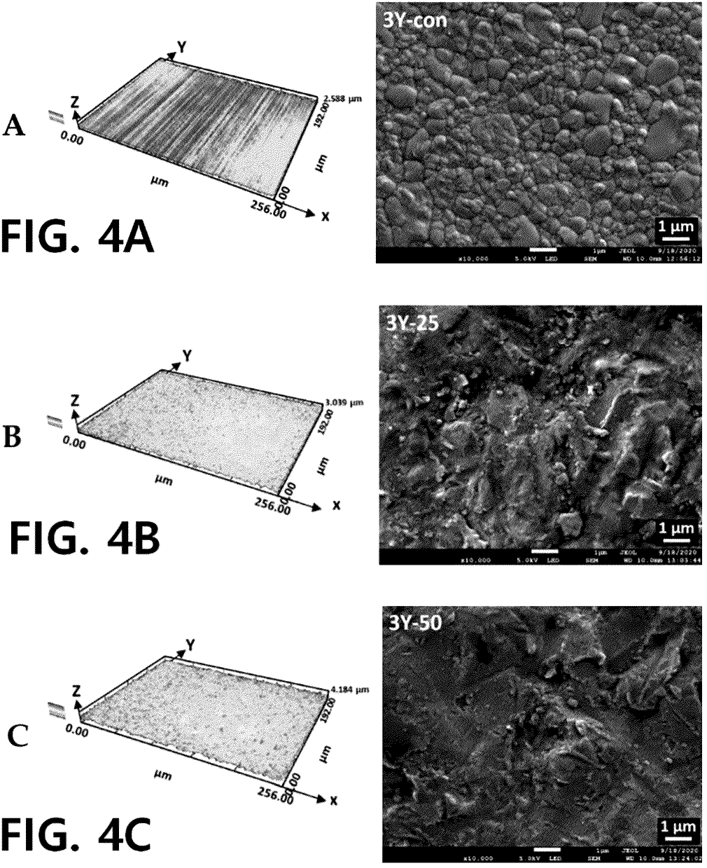

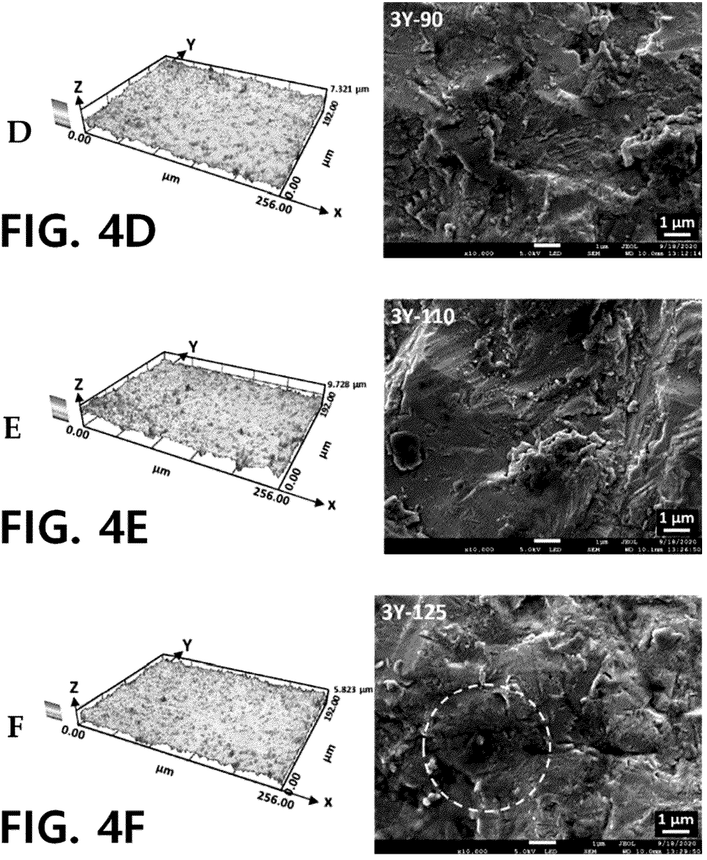

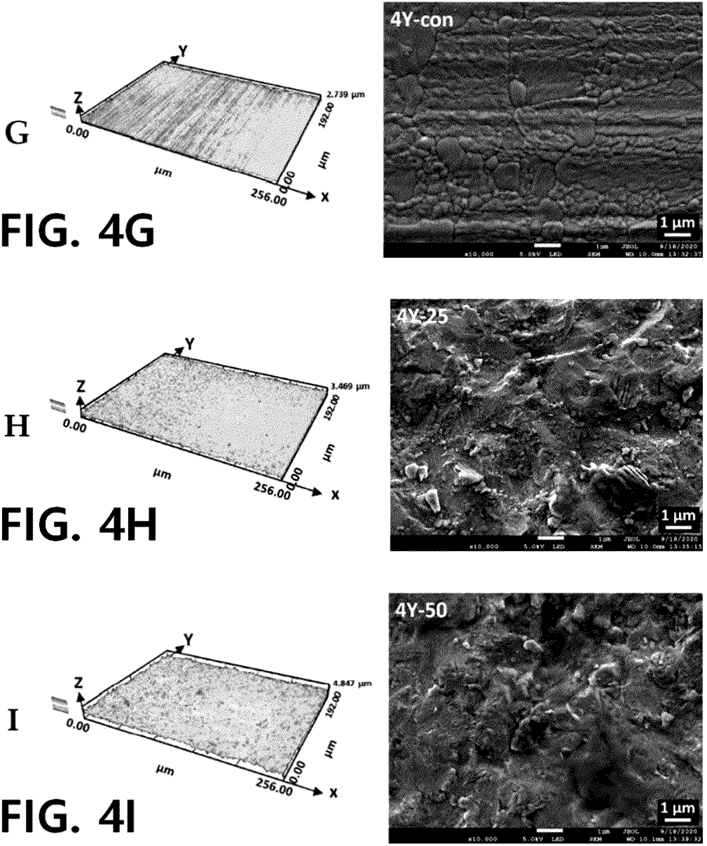

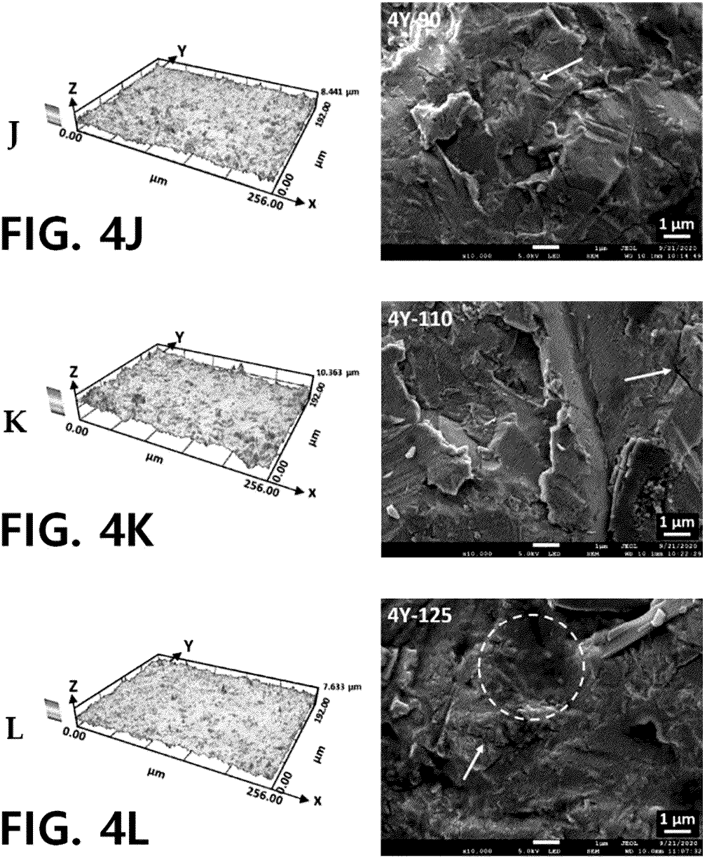

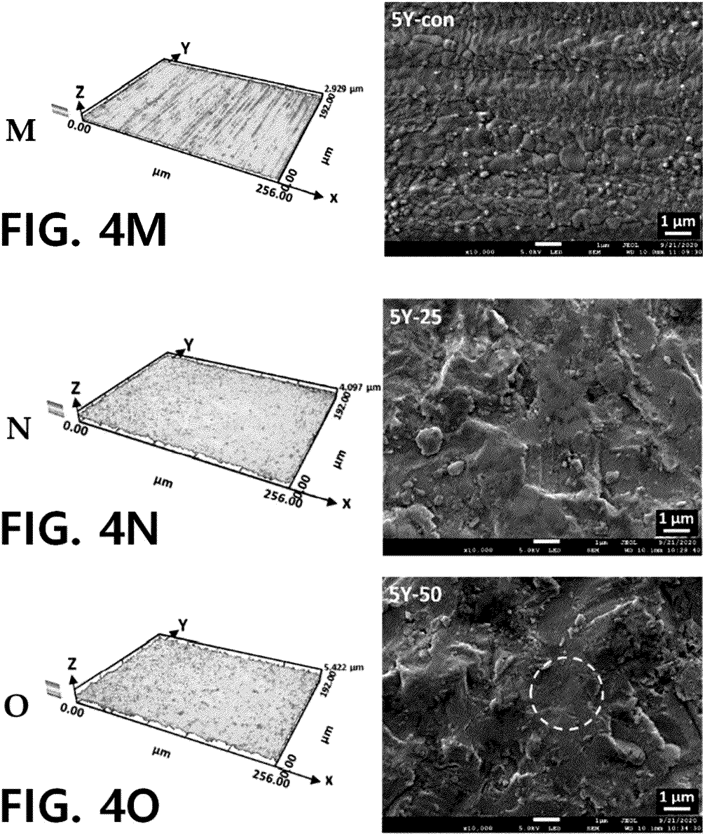

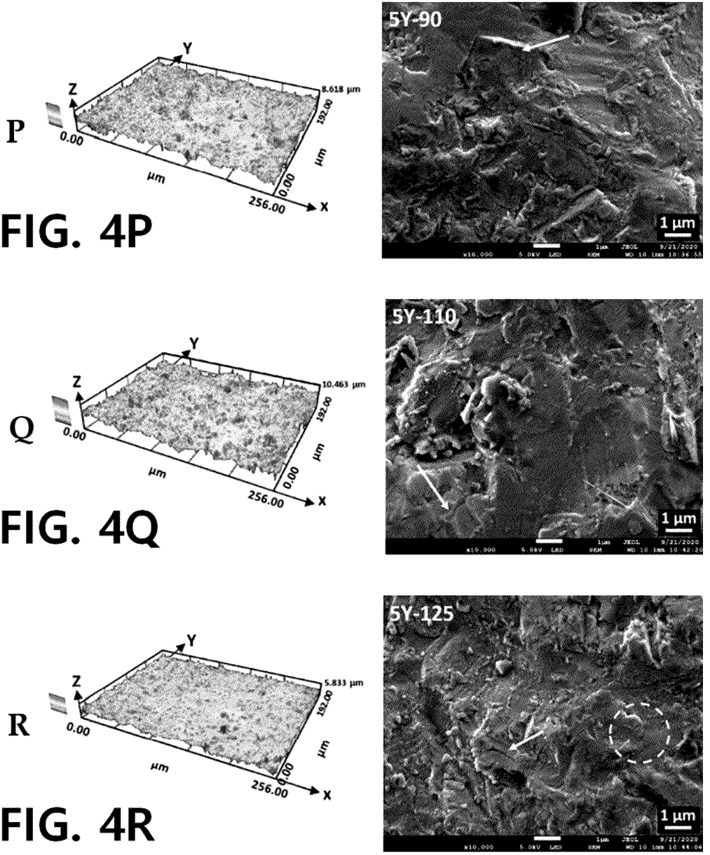

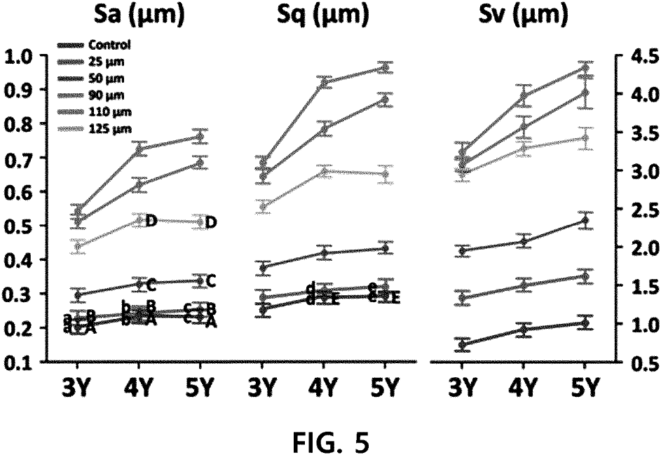

For 5Y-PSZ, larger particles made a bigger change in surface topography, and the change increased at a faster rate than other zirconia grades (FIG. 5). This finding may be attributed to the lowest flexural strength of 5Y-PSZ. The µRaman spectra in the present invention indicated that there is no obvious change in peak intensity after sandblasting because 5Y PSZ has little possibility of phase transformation of cubic particles (FIG. 6(C)). The tetragonal peak at 147 cm⁻¹ showed blue shift when treated with 25-µm sand (p < 0.05), indicating the presence of residual compressive stress. However, the µRaman peak shifted to a shorter wavelength after sandblasting with 50-µm or more sand (p < 0.05), which is referred to as red shift (FIG. 7). In contrast to the blue shift, a downshift of the Raman peak position occurs when the material is subjected to tensile strain. The red shift is related to the structural disorder associated with the decrease in phonon energy due to lattice defects or lattice stretching. In addition, an increased temperature may cause red shift due to increased thermal expansion in the crystal lattice. Accordingly, the plastic deformation and surface melting on the 5Y-PSZ surface observed in this study (FIG. 4) are caused by the internal tensile stress and increased temperature due to the impact load during sandblasting, resulting in structural degradation after sandblasting for 5Y-PSZ.

According to an exemplary embodiment of the present invention, when the zirconia is 5Y-PSZ, the average particle size of alumina particles may be 10 to 50 µm, and more preferably, 25 µm to prevent the possibility of surface damage.

In addition, according to the surface treatment method of the present invention, a target peak at 147 cm⁻¹ may shift to a higher wavenumber when sandblasting is not performed.

In the alumina particles of the present invention, trace elements of the alumina particles may include Si, Fe and Zr, which may be the same as specifically disclosed in Table 2 of Example 1 herein. However, other trace elements may be further included without limitation.

In the surface treatment method of the present invention, the surface treatment method may be for improving residual stresses.

According to an exemplary embodiment of the present invention, the surface treatment method may cause a bigger change in surface topography than that when the surface treatment is not performed.

In a specific embodiment, when the dental zirconia subjected to the surface treatment method of the present invention is 3Y-TZP, a larger blue shift may indicate the presence of a larger compressive stress, resulting in improved mechanical properties, and the use of 125 µm sand may lead to a decrease in surface roughness value, so 110 µm sand is most preferable. For 4Y-PSZ, a peak shifted to a higher wavenumber up to 90 µm sand, and since the creation of compressive stress enhanced the strength of sandblasted 4Y-PSZ using up to 90 µm sand to disturb the propagation of defects, 90 µm sand is most preferable. For 5Y-PSZ, the tetragonal peak at 147 cm⁻¹ showed blue shift when treated with 25 µm sand (p < 0.05), and because of the presence of residual compressive stress, 25 µm sand is most preferable.

The present invention provides a dental article including dental zirconia made by the surface treatment method of dental zirconia. Dental zirconia may be further processed, using commercially available dental CAD/CAM phases, into dental articles, such as dental restorations (blanks, full-contour fixed partial dentures (FPDs), bridges, implant bridges, multi-unit frameworks, abutments, crowns, partial crowns, veneers, inlays, onlays, occlusal braces, orthodontic spacing devices, tooth replacement, splints, dentures, posts, teeth, jackets, facing, occlusal facets, implants, cylinders and connections.

Hereinafter, the present invention will be described in more detail with reference to examples. The examples are merely provided to more fully describe the present invention, and it will be obvious to those of ordinary skill in the art that the scope of the present invention is not limited to the following examples.

### Preparation Example

### Preparation of Specimens

Dental zirconia materials having three different transparency levels investigated in this study are listed in Table 1.

TABLE 1

| Materials | Manufacturer | Shade | Batch No. | Sintering | Composition | Flexural strength (MPa) | Toughness (MP am½) |
|---|---|---|---|---|---|---|---|
| KATANA ML | Kuraray Noritake | A Light | EASLS | 1500° C. for 2 h | 3Y-TZP (15%c) | 900-1100 | 3.5-4.5 |
| KATANA STML | Kuraray Noritake | A2 | EAVHC | 1500° C. for 2 h | 4Y-PSZ (>25%c) | 600-800 | 2.5-3.5 |
| KATANA UTML | Kuraray Noritake | A2 | DZVML | 1500° C. for 2 h | 5Y-PSZ (>50%c) | 500-600 | 2.2-2.7 |

A total of 180 zirconia specimens were prepared in the form of sintered plates (14.0 mm × 14.0 mm × 1.0 mm, n = 60 for each grade). One side of the specimen was sequentially polished using an 800-grit silicon carbide paper under running water to ensure the same initial roughness, and then all specimens were thermally etched at 1400° C. for 30 minutes. The specimens of each zirconia grade were randomly divided into six subgroups consisting of 10 specimens. One of the subgroups of each zirconia grade was maintained as polished (untreated, control) and then other subgroups were sandblasted.

### Example 1

### Analyses of Al₂O₃ Particle Size, Shape and Chemical Components

Sandblasting was performed with commercially available Al₂O₃ particles of five different sizes (25, 50, 90, 110 and 125 µm; Cobra, Renfert, Hilzingen, Germany), and other parameters were maintained constant. A zirconia plate was placed on a customized specimen holder designed by the inventors (FIG. 1) 10 mm from the tip of a sandblasting apparatus equipped with a nozzle having a diameter of 2.0 mm. The specimen was then particle-polished using the sandblasting apparatus (Basic master, Renfert, Hilzingen, Germany) at an impact angle of 90° and a pressure of 0.2 MPa at 10 s/cm². Only the polished side was sandblasted. After sandblasting, all specimens were ultrasonically cleaned in 99.8% isopropanol at a frequency of 40 kHz for 20 minutes to remove Al₂O₃ particle debris from the zirconia surface, and then air-dried.

Since the quality of particle size distribution determined the significance and reliability of the analysis, the particle size distribution for the five sizes of Al₂O₃ blasting particles was measured through a wet granulation process using a laser scattering particle size analyzer (LSPSA; LA-350, HORIBA, Kyoto, Japan). Distilled water was used as a bonding liquid. The specific surface area of the investigated particles was measured for 10 seconds at intervals of 0.1 to 1000 µm.

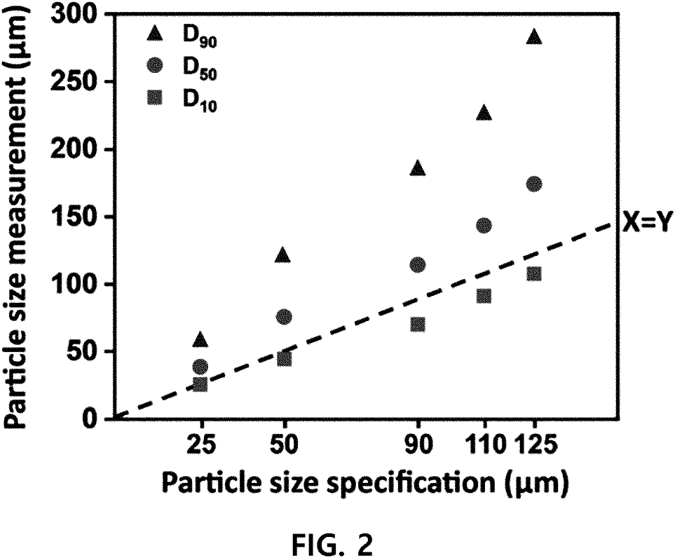

Consequently, as shown in FIG. 2, the Al₂O₃ particles showed five different specifications in particle size. There were considerable differences between all specifications, and the measured particle sizes increased in proportion to the increased specification (p < 0.05). The 125 µm specification showed the widest particle size distribution ranging from 29.91 to 592.39 µm, whereas the 25 µm specification showed the narrowest particle size distribution ranging from 13.25 to 116.2 µm.

The morphological images of the Al₂O₃ abrasive particles were acquired using scanning electron microscopy (SEM; JSM-IT500HR, JEOL, Tokyo, Japan) at 50×, 100×, or 200× magnification. The acceleration voltage of the negative electrode was set to 15 kV. All particles were coated with platinum (Pt) before SEM examination.

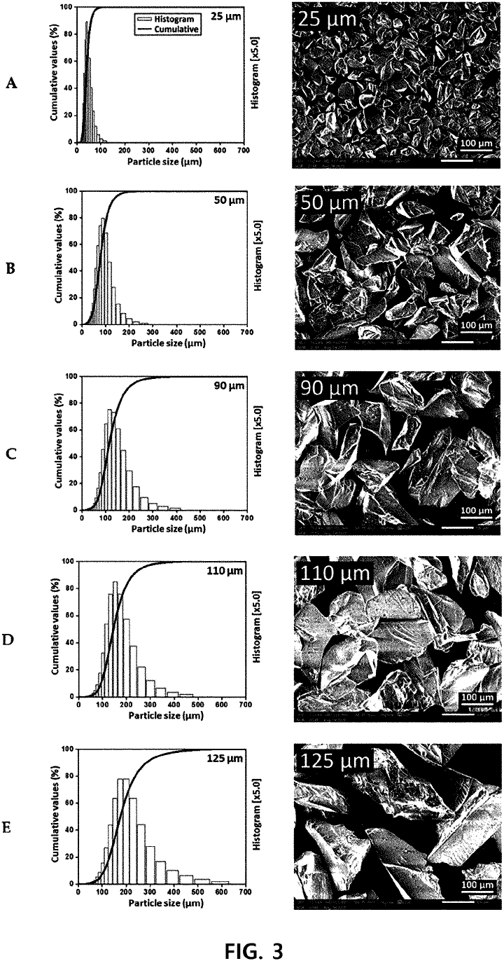

As a result, FIG. 3 shows the particle size distribution and microstructure of the Al₂O₃ particles. The SEM images showed that the finest particles were observed in the 25 µm specification (FIG. 3(A)), whereas the largest particles were prevalent in the 125 µm specification (FIG. 3(E)). Most of the particles had a grit shape with sharp and irregular edges, and some of the particles were spherical. The particles contained structural defects such as cracks and microcracks, and it was observed that the larger the particles, the greater the number of defects. It was observed that the particles of 125 µm specification were loosely packed, whereas the particles of 25 µm specification were uniformly distributed.

To confirm trace elements in the Al₂O₃ particles, inductively coupled plasma optical emission spectrometry (ICP OES; Agilent 5100, Agilent, Santa Clara, CA, USA) with a charge injection device (CID) detector was used. The specimens were introduced with a OneNeb Series 2 inert concentric nebulizer (Agilent, Santa Clara, CA, USA) and an inert double-pass spray chamber with a ball joint socket. The TOPEX microwave digestion system (PreeKem, Shanghai, China) was used for centrifugation.

As a result, Table 2 shows the concentrations of trace elements in the Al₂O₃ particles obtained by ICP OES. Si, Fe and Zr were detected at variable amounts. The 125 µm alumina sand showed the highest amounts of these elements with 545.963 ± 3.71, 80.348 ± 0.78, and 5.645 ± 0.01 mg/kg of Si, Fe and Zr, respectively.

TABLE 2

Trace element concentrations of Al₂O₃ particles obtained by ICP OES

| Element | 25 µM | 50 µM | 90 µM | 110 µM | 125 µM |
|---|---|---|---|---|---|
| Si | | | | | |
| Mean (mg/kg) | 259.971 | 322.217 | 356.979 | 173.206 | 545.963 |
| Standard deviation (mg/kg) | 0.39 | 1.87 | 0.61 | 0.76 | 3.71 |
| Relative standard deviation (%) | 0.15 | 0.58 | 0.17 | 0.44 | 0.68 |
| Limit of determination | | | 0.018 | | |
| Fe | | | | | |
| Mean (mg/kg) | 40.695 | 63.193 | 45.943 | 38.530 | 80.348 |
| Standard deviation (mg/kg) | 0.22 | 0.47 | 0.15 | 0.19 | 0.78 |
| Relative standard deviation (%) | 0.54 | 0.75 | 0.33 | 0.49 | 0.97 |
| Limit of determination | | | 0.002 | | |
| Zr | | | | | |
| Mean (mg/kg) | 1.657 | 3.270 | 3.684 | 1.842 | 5.645 |
| Standard deviation (mg/kg) | 0.03 | 0.04 | 0.03 | 0.03 | 0.01 |
| Relative standard deviation (%) | 1.82 | 1.21 | 0.54 | 1.53 | 0.03 |
| Limit of determination | | | 0.001 | | |

### Example 2

### Surface Topography Characterization

To analyze surface topography, CLSM and SEM were used after sandblasting, and area texture parameters were analyzed. Surface topography was investigated on the specifications of each subgroup using a 3D confocal laser scanning microscopy (CLSM; LEXT OLS3000, Olympus, Tokyo, Japan) at 50× magnification. The area texture parameters were obtained using software (LEXT-OLS, version 6.0.3, Olympus, Tokyo, Japan). Sa represents an arithmetic mean height; Sq represents a root mean square height; and Sv represents the maximum pit height of a scale-limited surface according to ISO 25178. Surface measurement was performed while morphology and outliers were removed. After correcting a tilt, a 3D surface was constructed with a distance to the optical center as the X axis, the tilt angle as the Y axis, and the flatness error as the Z axis. A powerful short-pass Gaussian filter (cutoff wavelength: 10 µm) was applied to data in order to decompose the wave shape from roughness. For each specimen, three different measurements (effective field of view was 256 × 192 µm) on either the polished sides for controls or the sandblasted sides for experimental subgroups were performed. A total of 30 measured values were obtained for each subgroup.

FIG. 4 shows the representative CLSM images and SEM microscopic images after sandblasting using different sizes of Al₂O₃ for three different dental zirconia grades, and FIG. 5 shows the values of Sa, Sq and Sv parameters of each subgroup.

Consequently, as shown in FIGS. 4 and 5, the surface roughness significantly increased with an increase in particle size up to 110 µm, whereas the surface roughness values decreased with the 125 µm alumina sand in all zirconia grades, lying between the that of the 50 µm alumina sand and that of the 90 µm alumina sand (p < 0.05). As a result of two-way ANOVA (p < 0.05), there was a statistically significant interaction between the Y₂O₃ amount and the Al₂O₃ particle size on the surface topography of the zirconia specimen. According to simple main effect analysis, Sa and Sq parameters were not significantly changed with 25 µm alumina sand in most subgroups (p > 0.05), and there were no significant differences in scalar values (Sa) among the non-polished subgroups (3Y-con, 4Y-con and 5Y-con) and the subgroups polished with 25 µm alumina sand (3Y-25, 4Y-25 and 5Y-25) (p > 0.05). For the sandblasted 4Y- or 5Y-PSZ, higher scalar values (Sa, Sq and Sv) were shown compared with those of the sandblasted 3Y-TZP. 5Y-PSZ polished with 110 µm sand showed the highest Sa value (0.76 ± 0.12 µm), the highest Sq value (0.97 ± 0.15 µm) and the highest Sv value (4.37 ± 0.16 µm). The Pearson correlation test revealed that there were positive correlations between the particle size (from the control up to 110 µm) and Sa, Sq or Sv parameters for all zirconia grades.
Sa; r = 0.930 (p < 0.001) for 3Y-TZP, r = 0.928 (p < 0.001) for 4Y-PSZ, and r = 0.891 (p < 0.001) for 5Y-PSZ,
Sq; r = 0.933 (p < 0.001) for 3Y-TZP, r = 0.930 (p < 0.001) for 4Y-PSZ, and r = 0.895 (p < 0.001) for 5Y-PSZ, or
Sv; r = 0.939 (p < 0.001) for 3Y-TZP, r = 0.949 (p < 0.001) for 4Y-PSZ, and r = 0.924 (p < 0.001) for 5Y-PSZ.

The surface microstructure was observed using a scanning electron microscope (JSM 7800F Prime, JEOL, Tokyo, Japan) equipped with energy-dispersive X-ray spectroscopy (EDX; Inca, Oxford Instruments, Abingdon, UK). One specimen randomly selected from each subgroup was treated by SEM at 3000× and 10,000× magnifications. The acceleration voltage of the negative electrode was set to 5.0 kV, and working distance (WD) was set to 10.0 mm. After the specimen was sputter-coated with gold, and secondary electron SEM images were then acquired under vacuum (10⁻⁵ mbar). The element composition of the specimen surface was analyzed using EDS.

As a result, the SEM images showed changes in surface morphology after sandblasting using various sizes of Al₂O₃ (FIG. 4). A grain boundary of the zirconia surface was observed in the sandblasted 3Y-TZP using 25 µm alumina sand, and the disappearance of grain boundaries was observed in all other sandblasted specimens. Surface damage such as microcracks, plastic deformation, surface melting and Al₂O₃ particle impact following an polishing process were detected.

In addition, the EDX analysis showed that the yttrium (Y) content increased with the increasing Y₂O₃ doping level of zirconia. After Al₂O₃ air polishing, the EDX analysis showed that the presence of aluminum (Al) was obvious on the zirconia surface (0.93-2.16%). The highest concentrations of Al for each zirconia grade were 2.01%, 2.13% and 2.16% for 3Y-90, 4Y-110 and 5Y-110, respectively.

### Example 3

### Quantitative Measurement of Zirconia Phases and Residual Stress

Micro-Raman spectroscopy (µRaman) was used to identify the phase transformation and surface residual stresses induced by sandblasting. Raman spectra (LabRAM HR Evolution, Horiba Scientific, Kyoto, Japan) were collected using a diode-pumped solid state laser (DPSSL) of 10 mV with a 532 nm wavelength through a 100× objective lens with a pinhole aperture of 50 µm.

The collection time of Raman scattering was 10 seconds, and two consecutive spectra were averaged. For each subgroup, 25 measured values were obtained. For quantitative determination of residual stresses in the specimens, the Raman wavenumber of a tetragonal phase zirconia (t-ZrO2) band at approximately 147 cm⁻¹ was tracked using curve fitting software (Origin 2020 Pro, OriginLab Corp., Northampton, MA, USA).

As a result, representative µRaman spectra are shown in FIG. 6. The spectrum shape of the 3Y-TZP specimen was clearly different from that of the 4Y- or 5Y-PSZ specimen. FIG. 7 shows the peak shift of the tetragonal band at approximately 147 cm⁻¹ due to stress induced on the zirconia surface after sandblasting as a function of Al₂O₃ particle size.

For 3Y-TZP, the tetragonal peaks at 147, 456 and 641 cm⁻¹ decreased, whereas the monoclinic peaks at 178 and 506 cm⁻¹ increased after sandblasting, and variances increased as the particle size increased (FIG. 6(A)). As shown in FIG. 7, the tetragonal peak at 147 cm⁻¹ shifted to a higher wavenumber after sandblasting (p < 0.05). The peak shift increased with increasing particle size up to 125 µm (p < 0.05). A protrusion was observed at the left part of the tetragonal peak for the sandblasted specimen, and the size of this peak increased with an increasing particle size.

For 4Y-PSZ and 5Y-PSZ, it was difficult to distinguish the tetragonal phase from the cubic phase due to the overlapping of wavenumbers at 147 cm⁻¹. As the particle size increased, the tetragonal peak at 641 cm⁻¹ decreased but the cubic peak at 625 cm⁻¹ was maintained (FIGS. 6(B) and 6(C)). The monoclinic peak at 178 cm⁻¹ slightly increased with an increasing particle size. As shown in FIG. 7, the tetragonal peak at 147 cm⁻¹ shifted to a higher wavenumber up to 90 µm sand for 4Y-PSZ (p < 0.05), whereas the peak shifted to a higher wavenumber up to 25 µm sand for 5Y-PSZ (p < 0.05).

Taken together, for 3Y-TZP, a larger blue shift may indicate the presence of larger compressive stress, resulting in improved mechanical properties, and since the surface roughness values decreased with 125 µm sand, 110 µm sand is most preferable. For 4Y-PSZ, a peak shifted to a higher wavenumber up to 90 µm sand, and since the creation of compressive stress enhanced the strength of sandblasted 4Y-PSZ using up to 90 µm sand and disturbed the propagation of defects, 90 µm sand is post preferable. For 5Y-PSZ, the tetragonal peak at 147 cm⁻¹ showed blue shift when treated with 25 µm sand (p < 0.05), and because of the presence of residual compressive stress, 25 µm sand is most preferable.

### Statistical Analysis

All tests were performed using software (IBM SPSS Statistics for Windows, v25.0, IBM Corp., Chicago, IL, USA) at a significance level α = 0.05. Normal distribution and the homogeneity of variances were verified by a Shapiro-Wilk test and a Levene test, respectively (p < 0.05). The statistically significant differences among the various blasting particle sizes were analyzed with one-way analysis of variance (ANOVA) followed by Tukey’s honestly significant difference (HSD) post-testing. Two-way ANOVA was performed on (1) surface topography and (2) the Raman wavenumber of the tetragonal phase zirconia (t-ZrO2) band at approximately 147 cm⁻¹ to determine the effect of two independent variables (yttria content, 3 mol% yttria for 3Y-TZP, 4 mol% yttria for 4Y-PSZ, 5 mol% yttria for 5Y-PSZ and an alumina particle size). The interaction between two independent variables was verified and pairwise comparison for simple main effects of the independent variables was analyzed using SPSS syntax. In addition, the Pearson correlations between the particle size and surface texture parameters of the subgroups for all zirconia grades were analyzed.

A surface treatment method according to the present invention can reduce the physical damage to a zirconia surface and promote bonding between resin cement and zirconia, thereby preventing detachment after a zirconia artificial tooth procedure. In addition, the method of the present invention can improve mechanical properties by strengthening the residual compressive stress of an artificial tooth through tetragonal-to-monoclinic phase transition of zirconia, and reduce the repair rate by peeling even after the procedure by increasing bond strength. Since the method of the present invention suggests the optimal alumina particle size and conditions for sandblasting of three types of dental zirconia, a clinically desired surface treatment method can be provided when each type of zirconia is used.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A surface treatment method for dental zirconia, comprising:
(a) polishing the surface of zirconia which mostly comprises tetragonal and cubic zirconia with less than 15% monoclinic phase, 95 vol% or more of all particles of which have an average diameter of 100 to 1200 nm, and which has a density of 99.5% or more of the theoretical density and is opalescent; and
(b) sandblasting the polished surface in (a) with alumina particles using a nozzle-equipped sandblasting apparatus.

2. The method of claim 1, wherein the zirconia in (a) is any one selected from the group consisting of 3 mol% yttria-stabilized tetragonal zirconia polycrystal (3Y-TZP), 4 mol% partially stabilized zirconia (4Y-PSZ), and 5 mol% partially stabilized zirconia (5Y-PSZ).

3. The method of claim 1, wherein, in (b), the vertical distance between the nozzle-equipped sandblasting apparatus and the polished surface is 1 to 100 mm.

4. The method of claim 1, wherein, in (b), the pressure for sandblasting with alumina particles is 0.1 to 0.5 Mpa.

5. The method of claim 1, wherein, when the zirconia is 3Y-TZP, the average size of the alumina particles is 100 to 120 µm.

6. The method of claim 1, wherein, when the zirconia is 4Y-PSZ, the average particle size of the alumina particles is 80 to 100 µm.

7. The method of claim 1, wherein, when the zirconia is 5Y-PSZ, the average particle size of the alumina particles is 10 to 50 µm.

8. The method of claim 5, wherein a target peak at 147 cm⁻¹ shifts to a higher wavenumber than that when sandblasting is not performed.

9. The method of claim 1, wherein the trace elements of the alumina particles comprise Si, Fe and Zr.

10. The method of claim 1, which is to enhance residual stress.

11. The method of claim 1, which causes a bigger change in surface topography than when surface treatment is not performed.

12. A dental article comprising dental zirconia made by the surface treatment method for zirconia of claim 1.

13. The method of claim 6, wherein a target peak at 147 cm⁻¹ shifts to a higher wavenumber than that when sandblasting is not performed.

14. The method of claim 7, wherein a target peak at 147 cm⁻¹ shifts to a higher wavenumber than that when sandblasting is not performed.
